(19) United States
(12) Patent Application Publication
(10) Pub. No.: US 2018/0211274 A1
(43) Pub. Date: Jul. 26, 2018

(54) Measurement of Health-Related Activity Using Sensors

(71) Applicant: Destiny Health, Inc., Bethesda, MD (US)

(72) Inventors: Adrian Gore, Houghton Estate (ZA); Alan Pollard, Tenafly, NJ (US); Stephen Ronald Mitchley, Darien, CT (US); Tal Gilbert, Westport, CT (US)

(73) Assignee: Destiny Health, Inc, Bethesda, MD (US)

(21) Appl. No.: 15/928,013

(22) Filed: Mar. 21, 2018

Related U.S. Application Data
(63) Continuation-in-part of application No. PCT/IB2016/055602, filed on Sep. 20, 2016.
(60) Provisional application No. 62/281,229, filed on Jan. 21, 2016.

(30) Foreign Application Priority Data
Sep. 21, 2015 (ZA) 2015/07033

(51) Int. Cl. G06Q 30/02; G16H 40/63

(52) U.S. Cl. CPC G06Q 30/0239; G06Q 50/01; G16H 40/63

(57) ABSTRACT

A system and method for measuring health-related activity from a participant. The method includes measuring a health-related activity of a participant including heart rate, active calories, and a number of steps from one or more wearable devices on the participant. Health-related activity information is displayed on a wireless handheld device to the participant. The device receives a defined health-related activity target with an associated discount to a scheduled payment owed by the participant for a good or a service. The following steps are periodically performed: i) accessing information on a scheduled payment being made for a product or service by the participant; ii) accessing data recorded by the one or more wearable sensors; iii) calculating a progress of the participant meeting the defined health-related activity target based on the data recorded and the defined health-related activity target; and iv) sending an update to the display with the progress.

### CROSS-REFERENCE TO RELATED APPLICATION

This patent application is a continuing application of PCT Application No. PCT/IB2016/055602, filed on Sep. 20, 2016, which claims priority from U.S. Provisional Patent Application No. 62/281,229, filed on Jan. 21, 2016, which claims priority from South African Patent application Ser. No. 2015/07033, filed Sep. 21, 2015, the entirety of each of the above reference applications are hereby incorporated by reference.

### BACKGROUND OF THE INVENTION

This patent application relates to a measurement of health-related activity using sensors.

### SUMMARY OF THE INVENTION

In accordance with one aspect of the invention a computer-based system and method for measuring health-related activity from a participant is disclosed. The method includes measuring a health-related activity of a participant including heart rate, active calories, and a number of steps from one or more wearable devices on the participant. A display of a wireless handheld device displays health-related activity information to the participant and receiving, on the wireless handheld device from the participant, a defined health-related activity target with an associated discount to a scheduled payment owed by the participant for a good or a service.

The following steps are periodically performed, for example, on a daily or weekly basis: i) accessing information on a scheduled payment being made for a product or service by the participant; ii) accessing data recorded by the one or more wearable sensors; iii) calculating a progress of the participant meeting the defined health-related activity target based on the data recorded and the defined health-related activity target that has been selected; and iv) sending an update to the display of the wireless handheld device with the progress which has been calculated.

In one example, the update further includes a discount to the scheduled payment in response to the defined health-related activity target being met.

In another example, the update further includes the progress which has been calculated, the defined health-related activity target, and the data recorded by the one or more wearable sensors.

In another example the one or more wearable sensors for measuring a participant's activity includes a geographic location of the participant.

In another example, the method further includes periodically performing accessing social media information to determine health-related activity; and calculating a progress of the participant meeting the defined health-related activity target based on the data recorded, the social media information, and the defined health-related activity target that has been selected. The social media information is may be verified by others in a group of social media information.

In another example, the method further includes periodically performing accessing participation in training by an employer or third party; and calculating a progress of the participant meeting the defined health-related activity target based on the data recorded the participation in the training, and the defined health-related activity target that has been selected.

In another example, the method further includes periodically performing accessing participation in a fitness program including a duration of the fitness program, a frequency of the fitness program, an amount of resistance provided by a machine in the fitness program, and maximum heart rate achieved during the fitness program; and calculating a progress of the participant meeting the defined health-related activity target based on the data recorded, the participation in the fitness program, and the defined health-related activity target that has been selected.

According to another aspect of the invention there is provided rewards method, the method including:
defining an activity target for a predetermined period;
receiving activity data including information relating to an activity that has been completed by a user during the predetermined period;
calculating a value of a periodic payment that the user needs to make for an article that the user has purchased; and
if the user meets the activity target for the predetermined period then reducing the value of the payment for that period.

The method may further include:
accessing historical activity data relating to a user;
using the historical activity data to determine the activity target for the user for the predetermined period;

The method may include transmitting a message to an external payment system to effect the payment for that period.

According to another aspect of the invention there is provided a rewards system, the system including:
a memory for storing therein user identification data identifying a user, activity target data including an activity target for the user and a value of a periodic payment that the user needs to make for an article that the user has purchased;
a communications module for receiving activity data including information relating to an activity that has been completed by the user and storing this in the memory together with previously stored activity data;
a processor adapted for calculating if the user meets the activity target for a predetermined period and if so then reducing the value of the payment for that period.

The processor may further transmit a message via the communications module including the user identity and the reward that has been awarded to the user.

According to another aspect of the invention there is provided a rewards method, the method including:
determining a wellness program level associated with a user;
calculating a value of a periodic payment that the user needs to make for an article that the user has purchased; and
if the wellness program level associated with the user is at or above a particular level during a predetermined period, then reducing the value of the payment for that period.

According to another aspect of the invention there is provided a rewards system, the system including:
a memory for storing therein user identification data identifying a user, data indicating a wellness program level associated with the user and a value of a periodic payment that the user needs to make for an article that the user has purchased; and
a processor adapted for calculating if the wellness program level associated with the user is at or above a particular level during a predetermined period and if so then reducing the value of the payment for that period.

### BRIEF DESCRIPTION OF THE DRAWINGS

FIG. 1 shows a schematic drawing of a system in accordance with an example embodiment;

FIG. 2 shows a view of a rewards display;

FIG. 3 shows a view of additional rewards display; and

FIG. 4 shows a view of system.

### DESCRIPTION OF PREFERRED EMBODIMENTS

In the following description, for purposes of explanation, numerous specific details are set forth in order to provide a thorough understanding of an embodiment of the present disclosure. It will be evident, however, to one skilled in the art that the present disclosure may be practiced without these specific details.

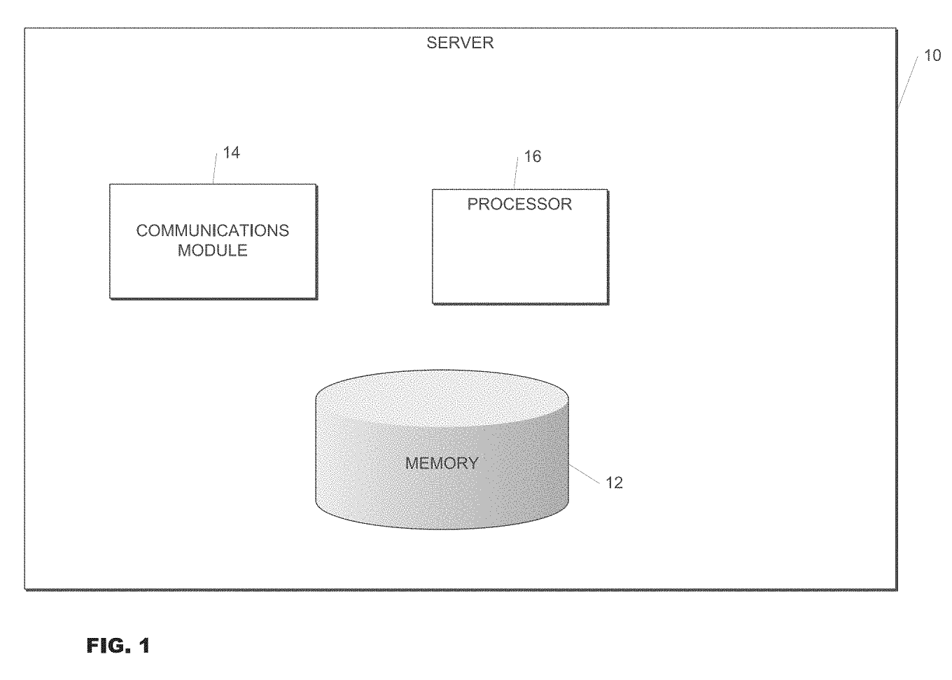

Referring to FIG. 1 of the drawings an example rewards system is generally indicated by reference numeral 10.

The system 10 in an example embodiment is at least one server including a number of modules for implementing the present invention.

The at least one server includes machine-readable instructions which are executable by the at least one server.

A memory in the form of a database 12 is connected to the server and is used for storing therein user identification data identifying a user, activity target data including an activity target for the user and a value of a periodic payment that the user needs to make for an article that the user has purchased.

A communications module 14 is used for receiving activity data including information relating to an activity that has been completed by the user and storing this in the memory together with previously stored activity data. This will be described in more detail below.

A processor 16 is adapted for calculating if the user meets the activity target for a predetermined period and if so then rewarding the user.

In one example, the reward is reducing the value of the payment for that period.

For purposes of illustration, the article that the user has purchased is an Apple Watch™.

The total cost of the watch is divided up into periodic payments, for example weekly or monthly.

The user is then given their activity target and the user's periodic repayment will be determined by the level of activity.

This could be set by the processor in a number of ways. In one simple example, if the user misses their activity target they will pay the full periodic payment whereas if the user meets their activity target they will pay a reduced or zero periodic payment.

In another example embodiment, the periodic payment could be adjusted in a more complex manner whereby the more active the user, the lower the payment ranging from zero to the full periodic payment.

The user's activity is measured in a number of different ways one of which is by a user wearable device. In the illustrated example, the watch itself has capability to monitor exercise such as walking or running. This data is captured and transmitted from the watch via a communications network to the communications module 14 of the server 10.

In addition, other activity data is collected from a system of a third party service provider such as gyms and other health partner visits. So for example, where the gym is operated by a third party, the gym will collect data relating to the number of attendances of the user and transmit this to the server 10.

Where the gym has machines that are able to monitor the user's actual activity and report this back to the server, this activity data is also captured and compiled.

In this example, the activity data could include a number of fields such as one or more of the duration of the activity, a speed at which the activity is performed, the amount of resistance provided by a machine on which the user is exercising, a height that the user reaches during the exercise, data relating to the user's heart rate during the activity and measurements of power to name a few examples.

In one example embodiment, the user's activities are allocated points which are added up to one or more predetermined threshold values. Each threshold value determines what the periodic payments will be. Obviously, the higher the threshold value the lower the periodic payment.

In one example embodiment, the activity target is specifically set for the individual by accessing historical activity data relating to the user stored in the memory 12.

This historical activity data is then used to determine the activity target for the user for a predetermined period.

An example of how this is accomplished is as follows. Each user is set a weekly physical activity goal that is both personalised and clinically relevant

Goals are based on the guideline recommendations for cardiovascular physical activity progression programs.

Goals are unique and personalised according to a member's key health and cardiovascular risks.

The following data is used to recommend both the weekly goals and underlying physical activity progression program:
1. Demographic information: Age, gender;
2. Biometric information: BMI (weight and height), including waist circumference;
3. Medical information: Smoking status, blood pressure, cholesterol, diabetes, heart disease, renal failure, cancer treatment; and
4. Current level of physical activity (as monitored within the Vitality program).

Using the above data, users are segmented into one of three health risk groups.

Each group is recommended activity goals based on the clinically appropriate cardiovascular progression program for that segment.

The goals can also be dynamically adjusted by an algorithm which combines the above information for each individual with their respective goal history (behaviour) to dynamically adjusted and recommend the subsequent weekly goals.

Any subsequent changes to a user's health and cardiovascular risk profile (from a variety of other data sources) are automatically fed through and used to dynamically adjust the recommended goal (level of activity).

It will be appreciated that a user that is not very active will be given a lower activity target than a user that is very active and in this manner each user will be motivated to improve their levels of activity and thereby improve their health.

In addition to add a level of “gamification” a random element can be added to the weekly points targets. This ensures that the target presented to the user is both clinically sound but also interesting in that some weeks it will be a stretch target and in others less onerous.

A digital display including the activity target generated for the user as well as an indicator of how the user is performing is generated and displayed to the user via the mobile communications device and/or the watch.

Typically the server will transmit data including the activity target as well as activity data indicating how the user is doing to the user's mobile communications device and/or the watch.

This is consolidated, summarised and presented back to users in the form of a single weekly activity goal.

The goals are expressed to a member in a single, consistent currency—Vitality fitness points.

Weekly goals are represented visually (as a circle showing ones progress towards the goal) on the user's mobile communications device and/or the watch and/or via website.

The user interface is dynamically updated to reflect the latest activity and progress towards the weekly goal.

At the end of each goal cycle (plus monitoring period)—it is clearly displayed whether the individual has completed his/her goal.

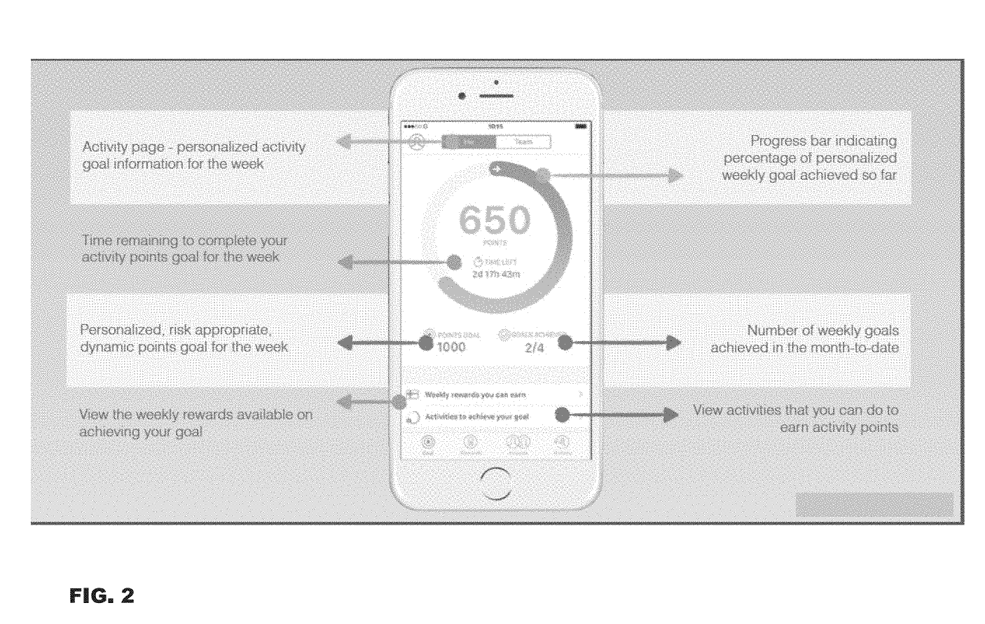

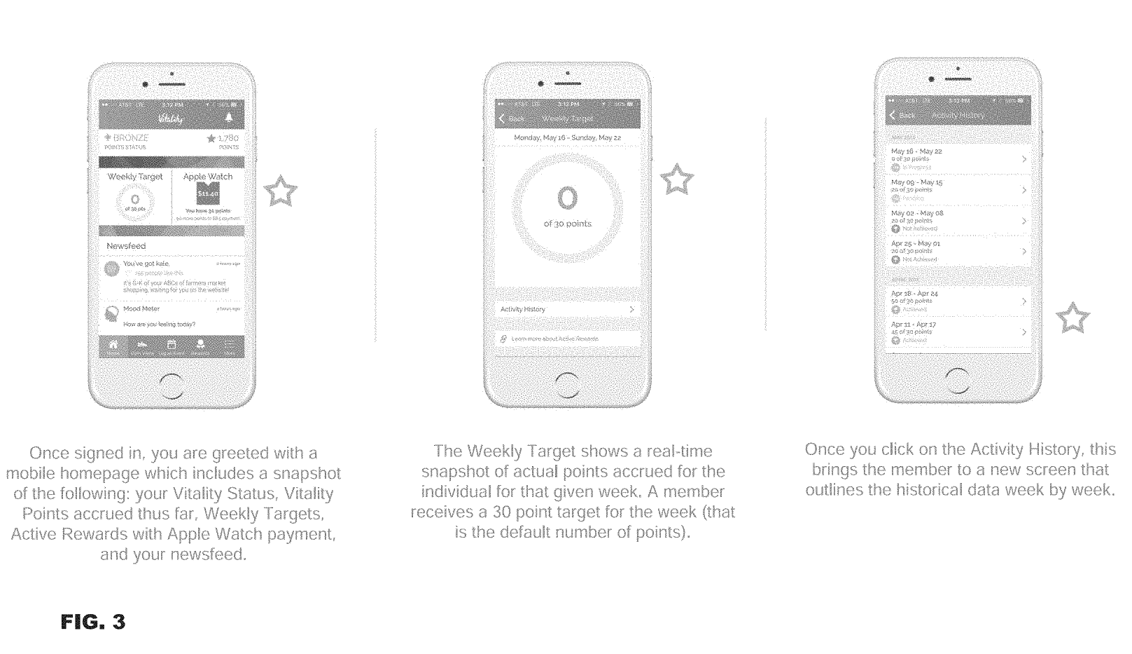

An example of the display is illustrated in FIG. 2. The display also includes an indication for the user of how much time they have remaining to complete their activity points goal. FIG. 3 shows a view of additional rewards display including from left to right
A defined health-related activity target, and data recorded by the one or more wearable sensors along with the product to which an associate discount to a scheduled payment owed by the participant is applied.
Periodic update of the first screen
Activity History

In the illustrated embodiment, the activity goal is a weekly goal whereas the periodic payment is a monthly payment. The user needs to meet their activity target every week to obtain the reduction in the monthly payment and therefore also included in the display is an indication of the number of weekly goals achieved in the month to date.

Also available for the user to display is a view of activities that they can do to earn activity points.

User can also invite up to four other users to form a team.

Such relationships are reciprocal i.e. if the invitation is accepted then user A is a team member to user B and vice versa.

Notwithstanding the above, each user can invite different users to join their network. This results in a spread of social of networks

Similarly—users can see the goal progress (displayed in the same way as the individual's goal above) of other users with whom they have agreed to form a team.

While each individual goal is personalised, the goal outcome of both the individual and each user in a team is aggregated (consolidated) in order to further incentivise and reward physical activity.

Typically however, in order for a user to receive a reward they based on their friend's goal achievement, the user must also meet their goal achievements.

In addition, a surprise rewards enhancement can be included whereby if, for example, 3 friends and the user achieve their weekly goals, the user will qualify for a surprise reward. The user will be guaranteed a reward, but that reward is probabilistic and the user will obtain a reward of different values according to set probabilities ranging from a coffee to a R250 voucher.

In addition to the above, the compliance of a person with a wellness program is monitored.

An example of such a wellness program is presently managed by the applicant and is disclosed in the applicant's South African granted patent number ZA 99/1746, the contents of which are incorporated herein by reference. The teachings of U.S. patent application Ser. No. 15/925,675 entitled “Dynamic Adjustment Of An Annuity Based On Wellness Monitoring Of A Participant”, filed Mar. 19, 2018 is hereby incorporated by reference in its entirety.

In an example wellness program, users participate in approved related facilities and/or services, for example gymnasiums, Smoke Enders™ and Weighless™.

Thus examples of compliance with a wellness program might include:
Compliance with a fitness program;
Weight loss program compliance;
Smoking cessation;
The purchase of healthy food products;
The compliance with drug regimes; and
Taking certain medical tests periodically.

In one example, users are awarded points and depending on the total number of points allocated to a user, they fall within one of five statuses. In the implementation of the wellness program, these statuses have been named in ascending order of value, blue, bronze, silver, gold and diamond. All users are placed initially in the blue status. Once a person has accumulated a predetermined number of points, his/her status is upgraded to the next appropriate level.

It will be appreciated that for purposes of the present invention the wellness program could be operated by a third party that operates the wellness program and then informs a product seller of the degree of participation of the person with the wellness program.

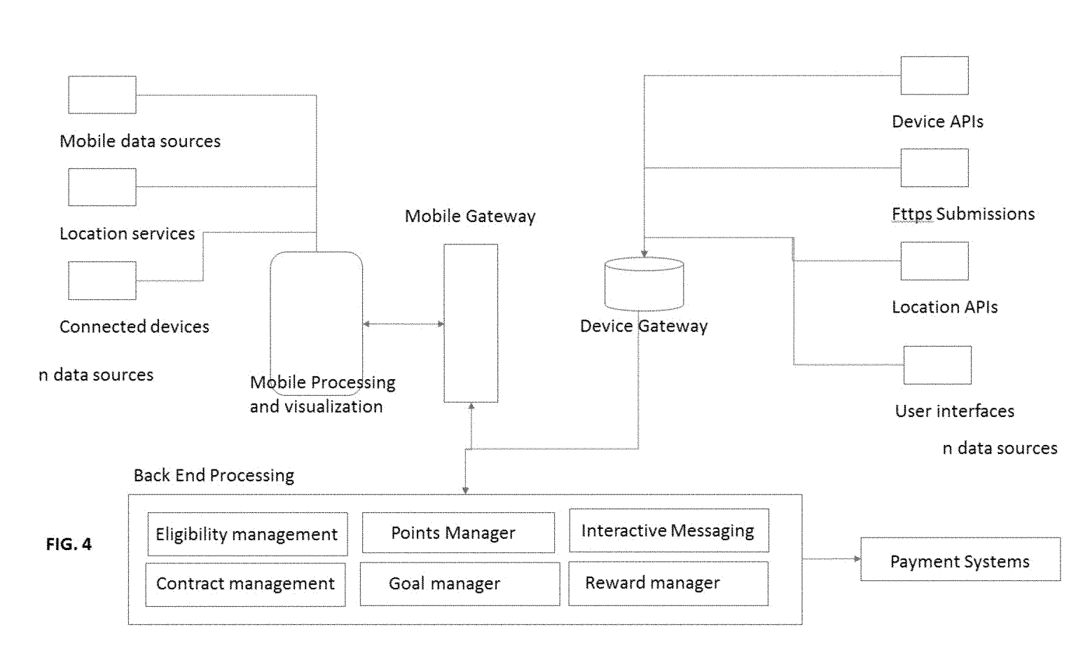

This could occur by a third party transmitting data to at least one server operated by the product seller. The data is received at the server 10 and stored in the database/memory 12 associated with the at least one server. Many of these functions are part of the back end processing as shown in FIG. 4.

In one example the data will include at least an identity of the person and data identifying the status of the person, for example blue, bronze, silver, gold and diamond.

In order to determine the status, a monitoring of the compliance of the person with the wellness program includes receiving usage information associated with the person's usage of the wellness program and determining, based on the usage information, a status level associated with the person for the wellness program, wherein the status level indicates a degree of participation in the wellness program

The usage information received may include information associated with a plurality of separate and distinct occurrences of wellness program participation by the person.

The usage information can then be used to determine a payback as will be described below.

In an alternative embodiment, the method may further include determining a status level associated with the person that further includes receiving the usage information from an information processing system and assigning a given point value to each separate and distinct occurrence of wellness program participation.

A total point value is determined based on an accumulation of each given point value assigned to each separate and distinct occurrence of wellness program participation

The total point value is then compared to a plurality of point ranges, each point range in the plurality of point ranges being associated with a status level and identifying a point range in the plurality of point ranges corresponding to the total point value.

Finally, a status level is assigned that is associated with the point range that has been identified to the person.

Data relating to the compliance of the person with the wellness program is stored in the memory/database 12.

In one example embodiment, the stored wellness data is used as one factor to calculate the amount of a periodic payment.

In one example, wellness program data relating to the compliance of a person with a wellness program is received and stored. Reference is now again made to the situation where periodic payments should be made by a user for an article that the user has purchased. In this case, the processor 16 may be adapted to reduce the value of the payment for a particular period by a certain amount, based on a current point value or status level associated with the person within the wellness program. The reduction value can typically be made dependent on the status level of the person such that the higher the status, the less the user needs to pay for that particular period. The table below provides an example of the reductions which can be provided:

| | Percentage Reduction |
|---|---|
| Status | (%) |
| Blue | 0% |
| Bronze | 10% |
| Silver | 20% |
| Gold | 30% |
| Diamond | 40% |

If, for example, the article that the user has purchased is an Apple Watch™ and amounts to a required payment of R500 per month, then he will only need to pay R350 per month instead, if he has a gold status. If, at some stage, his status changes to diamond, then he will only need to pay R300 per month.

It will be appreciated that the reduction of the periodic payments provided as a result of the activity targets and the status level associated with a person within the wellness program, may be combined in that the reduction is dependent on both the status of the person and whether or not his activity targets have been met.

By again using a similar example as described above, if a person needs to pay R500 per month for an Apple Watch™, but reaches all his weekly activity targets, then he may not need to pay anything for that particular month. If he reaches only part of which weekly targets, then he may qualify for a lesser reduction (e.g. 20%). However, if he has a gold status, then he may obtain a further 30% discount. In other words, the repayment may be reduced by (1) meeting certain activity targets and (2) having a certain status (as described above).

Tiering may alternatively or in addition be applied by factors other than Vitality status e.g. geographic area; based on the history of past weekly goals achieved; based on the underlying insurance product that the member has; or base on your employer grouping.

The combination of the activity targets and the compliance with the wellness program may be used to calculate other rewards.

In this example, the combination of the person's wellness program status together with whether or not they have met their activity targets will be used to determine the reward.

For example, the higher the person's wellness program status the higher the reward which could then be reduced and/or increased if the person has met their activity targets.

For example, if a wellness programme member is rewarded with a percentage discount on their monthly gym membership, this percentage discount will be increased if they meet their activity target in the previous month. Thus a 75% reduction in a monthly gym membership payment could become a 100% reduction in the payment if the previous month's activity targets are met.

Another non-limiting example is a wellness programme member will receive between 15%-35% discount when purchasing flights based on their wellness status. This discount will be increased by 5% if they meet their activity target.

One example implementation of this is that when the member books the flight they receive their discount based on their wellness status. If they then meet their activity target between the time of booking the flight and when they actually take the flight, this discount will be increased.

Turning now are details on various components of the system as shown in FIG. 4.

1) Qualifying Activity
The member or participant is awarded points based on measured physical thresholds. These thresholds are set at an individual level based on the member's personal data and may include gender, age and weight and clinical risk.
Heart rate is 60% of maximal heart rate by sex over a 30 min period. Active calories are normalized to equivalent step counts adjusted for weight and gender. Above give flexibility to adjust or add parameters.

2) Sources of Data

Physical activity is measured through a range of devices and means. These include:
Step and activity measurement on mobile devices, such as smart phones, tablets, and dedicated activity trackers such as Fitbit
Heart rate, calorie expenditure, steps and activity on smart watches
Steps, calories or heart rate on wearable devices for tracking user health, fitness, location, and biofeedback
Location based services to determine presence at fitness facilities
Kiosks and access control systems at fitness facilities
Use of Beacons or near field technology to verify proximity to equipment such as a rowing machine
Cameras with object and facial recognition to verify attendance or participation
Data feeds from sporting event providers
Social verification of activity in group settings
Employer, trainer or coaching input via a web or mobile interface/website or file submission
Fitness apps

Data can be sourced in real time on mobile devices or retrospectively through API integration with a network of partners through communication module 14 and memory 12 of FIG. 1. This data may include social media information and information verified through others in a defined group of friends or contacts on social media. For example, a user may have claimed to hike. Another participant on the hike could verify the user's participation.

In another example facial recognitions along with geographic location of the user can be used to infer activity. For example, the user may be inferred to be part of a running race or fitness class based on the combination of facial recognition and the location of the event measure by the sensor.

3) Experience
The participant experience seeks to inform the member on progress toward their daily target and weekly goals as well as framing rewards that are possible for this achievement. The Member is motivated to interact through making a visual link between goal attainment and monthly earnings toward the product or service.

4) Periodic Targets
Periodic targets attained through daily point earnings are set based on an algorithm that takes into account and adjusts for previous goal attainment and progress on the program. The algorithm dynamically adjusts weekly and may include some randomization and risk adjustment for defined cohorts.

The modules described above may be implemented by a machine-readable medium embodying instructions which, when executed by a machine, cause the machine to perform any of the methods described above.

It will be appreciated that embodiments of the present invention are not limited to such architecture, and could equally well find application in a distributed, or peer-to-peer, architecture system. Thus the modules illustrated could be located on one or more servers operated by one or more institutions.

In another example embodiment the modules may be implemented using firmware programmed specifically to execute the method described herein.

It will also be appreciated that in any of these cases the modules form a physical apparatus with physical modules specifically for executing the steps of the method described herein.

What is claimed is:

1. A computer-based system for measuring health-related activity, the computer-based system comprising:
one or more wearable sensors for measuring health-related activity of a participant including heart rate, active calories, and a number of steps;
a display on a wireless handheld device to display health-related activity information to the participant and to receive, on the wireless handheld device from the participant, a defined health-related activity target with an associated discount to a scheduled payment owed by the participant for a good or a service; and
a hardware processor to execute program instructions to periodically perform
accessing information on a scheduled payment being made for a product or service by the participant;
accessing data recorded by the one or more wearable sensors;
calculating a progress of the participant meeting the defined health-related activity target based on the data recorded and the defined health-related activity target that has been selected; and
sending an update to the display of the wireless handheld device with the progress which has been calculated.

2. The computer-based system of claim 1, wherein the sending the update further includes a discount to the scheduled payment in response to the defined health-related activity target being met.

3. The computer-based system of claim 1, wherein the one or more wearable sensors for measuring a participant's activity includes a geographic location of the participant.

4. The computer-based system of claim 1, wherein the hardware processor to execute program instructions to periodically perform further includes:
accessing social media information to determine health-related activity; and
calculating a progress of the participant meeting the defined health-related activity target based on the data recorded, the social media information, and the defined health-related activity target that has been selected.

5. The computer-based system of claim 4, wherein the accessing social media information to determine health-related activity includes verification by others in a group of social media information.

6. The computer-based system of claim 1, wherein the hardware processor to execute program instructions to periodically perform further includes:
accessing participation in a training by an employer or third party; and
calculating a progress of the participant meeting the defined health-related activity target based on the data recorded, the participation in the training, and the defined health-related activity target that has been selected.

7. The computer-based system of claim 1, wherein the hardware processor to execute program instructions to periodically perform further includes:
accessing participation in a fitness program including a duration of the fitness program, a frequency of the fitness program, an amount of resistance provided by a machine in the fitness program, and maximum heart rate achieved during the fitness program; and
calculating a progress of the participant meeting the defined health-related activity target based on the data recorded, the participation in the fitness program, and the defined health-related activity target that has been selected.

8. The computer-based system of claim 1, wherein the hardware processor to execute program instructions to periodically perform is performed daily.

9. The computer-based system of claim 1, wherein the hardware processor to execute program instructions to periodically perform is performed weekly.

10. The computer-based system of claim 1, wherein the sending an update to the display of the wireless handheld device with the progress which has been calculated, the defined health-related activity target, and the data recorded by the one or more wearable sensors.

11. A computer-implemented method for measuring health-related activity, the computer-implemented method comprising:
measuring a health-related activity of a participant including heart rate, active calories, and a number of steps from one or more wearable devices on the participant;
displaying, on a display of a wireless handheld device, health-related activity information to the participant and receiving, on the wireless handheld device from the participant, a defined health-related activity target with an associated discount to a scheduled payment owed by the participant for a good or a service; and
periodically performing
accessing information on a scheduled payment being made for a product or service by the participant;
accessing data recorded by the one or more wearable sensors;
calculating a progress of the participant meeting the defined health-related activity target based on the data recorded and the defined health-related activity target that has been selected; and
sending an update to the display of the wireless handheld device with the progress which has been calculated.

12. The computer-implemented method of claim 11, wherein the sending the update further includes a discount to the scheduled payment in response to the defined health-related activity target being met.

13. The computer-implemented method of claim 11, wherein the one or more wearable sensors for measuring a participant's activity includes a geographic location of the participant.

14. The computer-implemented method of claim 11, wherein periodically perform further includes:
accessing social media information to determine health-related activity; and
calculating a progress of the participant meeting the defined health-related activity target based on the data recorded, the social media information, and the defined health-related activity target that has been selected.

15. The computer-implemented method of claim 14, wherein the accessing social media information to determine health-related activity includes verification by others in a group of social media information.

16. The computer-implemented method of claim 11, wherein the periodically performing further includes:
accessing participation in a training by an employer or third party; and
calculating a progress of the participant meeting the defined health-related activity target based on the data recorded, the participation in the training, and the defined health-related activity target that has been selected.

17. The computer-implemented method of claim 11, wherein the periodically performing further includes:
accessing participation in a fitness program including a duration of the fitness program, a frequency of the fitness program, an amount of resistance provided by a machine in the fitness program, and maximum heart rate achieved during the fitness program; and
calculating a progress of the participant meeting the defined health-related activity target based on the data recorded, the participation in the fitness program, and the defined health-related activity target that has been selected.

18. The computer-implemented method of claim 11, wherein the periodically performing is performed daily.

19. The computer-based system of claim 11, wherein the periodically performing is performed weekly.

20. A computer program product for measuring health-related activity, the computer program product comprising:
a non-transitory storage medium, readable by a processing circuit and storing instructions for execution by the processing circuit for performing a method comprising
measuring health-related activity of a participant including heart rate, active calories, and a number of steps from one or more wearable devices on the participant;
displaying, on a wireless handheld device, health-related activity information to the participant and receiving, on the wireless handheld device from the participant, a defined health-related activity target with an associated discount to a scheduled payment owed by the participant for a good or a service; and
periodically performing
accessing information on a scheduled payment being made for a product or service by the participant;
accessing data recorded by the one or more wearable sensors;
calculating a progress of the participant meeting the defined health-related activity target based on the data recorded and the defined health-related activity target that has been selected; and
sending an update to the display of the wireless handheld device with the progress which has been calculated.
